(19) United States
(12) Patent Application Publication
(10) Pub. No.: US 2015/0031740 A1
(43) Pub. Date: Jan. 29, 2015

(54) COSMETIC COMPOSITION FOR STIMULATING THE CELLULAR ANTI-AGING FUNCTIONS OF THE SKIN

(71) Applicants: SYNTIVIA, Toulouse Cedex 1 (FR); CENTRE NATIONAL DE LA RECHERCHE SCIENTIFIQUE, Paris (FR); UNIVERSITE PUAL SABATIER TOULOUSE III, Toulouse (FR)

(72) Inventors: Philippe Bedos, Donneville (FR); Michel Baltas, Castanet Tolosan (FR)

(21) Appl. No.: 14/364,960

(22) PCT Filed: Dec. 14, 2012
(86) PCT No.: PCT/EP2012/075524

§ 371 Date: Jun. 12, 2014

(30) Foreign Application Priority Data
Dec. 16, 2011 (FR) 1161844

(51) Int. Cl. A61K 8/49; A61Q 19/08

(52) U.S. Cl. CPC A61K 8/492; A61Q 19/08; A61K 2800/10
USPC 514/419

(57) ABSTRACT

The use of a compound of general formula (I), where: R₁, R₂, R₃, R₄, R₅ are identical or different and each represent a hydrogen atom, a halogen atom, a hydroxyl group or an —OR′ radical in which R′ is a straight or branched, saturated or unsaturated C1-C16 hydrocarbon radical, wherein at least one group from among R₁, R₂, R₃, R₄, and R₅ does not represent a hydrogen atom or a halogen atom; X is a halogen atom, a hydroxyl group, a nitro group, a straight or branched, saturated or unsaturated optionally substituted C1-C14 hydrocarbon radical, or an —OR″ radical in which R″ is a straight or branched, saturated or unsaturated optionally substituted C1-C14 hydrocarbon radical; and Z is a covalent bond or a spacer arm, or of one of the salts thereof, in a cosmetic composition for stimulating the cellular anti-aging functions of the skin.

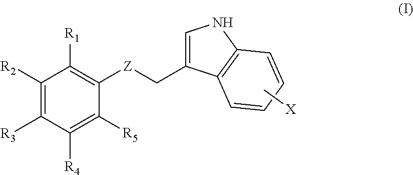

The present invention lies in the field of anti-aging cosmetic compositions. More particularly, it relates to the use of a compound corresponding to a particular general formula, in a cosmetic composition, for preventing and repairing the effects of aging of the skin, and more particularly for stimulating the anti-aging cellular functions of the skin.

The skin is the first barrier protecting the body against external attacking factors. The phenomenon of aging of the skin, whether it is natural or induced by these external attacking factors, weakens this barrier function. The symptoms of this aging are especially the appearance of wrinkles, dryness, roughness, thinning and loss of elasticity of the skin. Treatment of these symptoms has become a major challenge for the cosmetic industry.

However, although the knowledge of skin physiology has made it possible to propose cosmetic solutions to various dysfunctions induced by external attacking factors, no satisfactory solution has been proposed at the present time by the prior art for preventing and repairing the effects of aging of the skin.

Among the main cosmetic active agents proposed by the prior art for improving the appearance of the skin, examples that may be mentioned include compounds of plant origin such as polyphenols, whose antioxidant activity has been exploited for preventing oxidative stress in the skin, by trapping free radicals resulting from external attacking factors, for example exposure of the skin to ultraviolet (UV) rays. Caffeic acid is an example of such a polyphenol used in “anti-aging” cosmetic compositions proposed by the prior art. Cosmetic compositions based on such antioxidant compounds make it possible, to a certain extent, to slow down the appearance of the symptoms of aging. However, their efficacy is limited, and they also do not make it possible to repair the effects of aging.

The present invention is directed toward overcoming the drawbacks of the “anti-aging” cosmetic compositions proposed by the prior art, especially those mentioned above, by proposing such a composition which can efficiently combat the effects of aging of the skin, by preventing, and even repairing, the symptoms thereof.

It has now been discovered by the present inventors that a certain class of compounds, corresponding to a particular general formula, can achieve such a result when used as active principles in cosmetic compositions.

The present invention thus relate to the use of a compound of general formula (I):

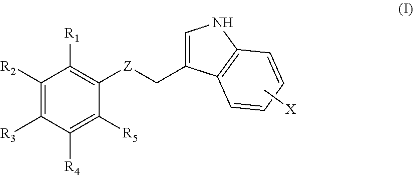

in which:

R₁, R₂, R₃, R₄, R₅, which may be identical or different, each represent a hydrogen atom, a halogen atom, such as a fluorine, chlorine, bromine or iodine atom, a hydroxyl group or a radical —OR′ in which R′ represents a linear or branched, saturated or unsaturated C1-C16 hydrocarbon-based radical, at least one group from among R₁, R₂, R₃, R₄ and R₅ not representing a hydrogen atom or a halogen atom,

X represents a halogen atom, a hydroxyl group, a nitro group, a linear or branched, saturated or unsaturated, optionally substituted C1-C14 hydrocarbon-based radical, or a radical —OR″ in which R″ represents a linear or branched, saturated or unsaturated, optionally substituted C1-C14 hydrocarbon-based radical,

Z represents a covalent bond or a spacer arm,

or a salt thereof,

in a cosmetic composition for stimulating the anti-aging cellular functions of the skin.

The expression “stimulating the anti-aging cellular functions of the skin” means that the compounds according to the invention act on skin cells, to stimulate the natural functions thereof that are involved in the mechanisms for combating the symptoms of aging of the skin and for repairing these symptoms. More particularly, it has been discovered by the present inventors that the compounds according to the invention act on the expression of genes involved in these mechanisms, in a sense promoting protection against the effects of aging of the skin, and even repairing the symptoms associated therewith. Thus, the compounds according to the invention, when applied topically to the skin, induce the overexpression of certain genes of skin cells coding for proteins with a cutaneous anti-aging effect, and the inhibition of expression of other genes coding for proteins that bring about symptoms of aging. These compounds, when placed in contact with skin cells, especially have the effect of inducing an increase in the production, by these cells, of proteins with a cutaneous anti-aging effect, such as collagen. The action of the compounds according to the invention is especially expressed by a reinforcement of the barrier function of the skin, the induction of neosynthesis of essential proteins of the cutaneous extracellular matrix, and the activation of regenerating or detoxifying proteins in the skin.

As genes whose overexpression is induced by a compound according to the invention, examples that may be mentioned include:
the gene coding for sirtuin 5, a mitochondrial enzyme which increases cell survival by retarding apoptosis, more particularly by deacetylation of cytochrome C, a protein which plays a central role in the induction of apoptosis (Schlicker et al., 2008). The overexpression of this enzyme promotes cell survival.
the gene coding for the protein Klotho, a hormone having the capacity of increasing cell survival, via its involvement in combating oxidative stress, regulating cell growth and regulating the ion channels (Kuro et al., 2009).
genes involved in the differentiation and adhesion of keratinocytes, which are included in the constitution of the epidermis, the layer of the skin which mainly affords its barrier function. With age, this barrier weakens, leading to embrittlement and dehydration of the epidermis. The cohesion of the epidermis is ensured by several intercellular adhesion structures. Over time, this cellular cohesion slackens, affecting the barrier function and the leaktightness of the skin. An increase in the expression of proteins involved in the differentiation and adhesion of keratinocytes promotes the formation and leaktightness of the skin barrier, which makes it possible, firstly, to maintain the moisturization of the skin, and, secondly, to protect the skin against external attacking factors and to combat its aging. Such genes are especially the genes coding for Epiplakin 1 (EPPK1), a protein associated with the intermediate filaments (IF) of keratin and of vimentin, which participates in maintenance of the cytoskeleton during the differentiation of keratinocytes (Jang et al., 2005); Envoplakin (EVPL), which participates in the formation of the desmosomes, in which it allows the binding of the intermediate filaments (IF) of keratin (Karashima et al., 2002); Transglutaminase 1 (TGM1), an enzyme capable of catalyzing the binding between precursors of the cornified envelope and the cell membrane, playing a key role in differentiation, and promoting the formation of the skin barrier and moisturization of the skin (Eckert et al., 2005); serine peptidase inhibitor, Kazal type 5 (SPINK5), a desquamation-inhibiting protein, expressed in corneocytes and participating in maintaining the cornified layer and moisturization of the skin (Chavanas et al, 2000); aquaporin 3 (AQP3), a specific intermembrane molecule of the epidermis, which forms, in the cell wall, tunnels specialized in the passage of water, making it possible to conserve hydric equilibrium in the skin, even under unfavorable conditions, the impairment of its metabolism, especially caused by age, bringing about a disruption in the hydric gradient reflected by poor circulation of water (Hara-Chikuma et al., 2005); cornulins (CRNN), which are terminal differentiation markers, also known as Sepp53 (squamous epithelial heat shock protein 53) (Contzler et al., 2005); keratin 1 (KRT1), a protein participating in the formation of the cornified envelope and the barrier function (Senshu et al., 1996).
genes coding for proteins of the extracellular matrix (ECM). The extracellular matrix consists of numerous proteins which make the skin firm and tonic while remaining elastic. The proteins most widely known are collagens for their sheathing power, elastin with elastic properties and hyaluronic acid for its structuring effect. Over time, these structural proteins are no longer renewed and end up by being degraded, which brings about a loss of elasticity, destructuring of the skin and the appearance of wrinkles. In particular, the compounds according to the invention are capable of increasing the expression of genes of the ECM and of promoting the neosynthesis of these structural proteins. Such genes are especially the genes coding for collagens 1, 3 and 5 (COL1A1, COL3A1, COL5A1), which are essential structural proteins of the ECM, which also have the function of giving the skin mechanical strength on stretching (Tzaphlidou, 2004, Kuo et al., 1997); elastin (ELN), an elastic protein of the ECM which gives the skin elasticity (Uitto et al., 1979); laminin alpha 5 (LAMAS), which promotes cellular adhesion at the dermo-epidermal junction and maintenance of the skin integrity (Schneider et al., 2006); hyaluronan synthase 3 (HAS3), an enzyme responsible for the synthesis of hyaluronic acid, an essential constituent of the ECM responsible, inter alia, for moisturization of the skin (Manuskiatti et al., 1996); secreted protein acidic cystein-rich, or osteonectin (SPARC), a protein expressed in permanently-renewed tissues, such as the skin and at sites of trauma or remodeling, which binds to various constituents of the extracellular matrix (fibrillar collagens, collagen IV, thrombospondin-1, vitronectin, etc.) and participates in its organization (Bradshaw et al., 2002).

As genes whose expression would be inhibited by a compound according to the invention, examples that may be mentioned include the gene coding for matrix metalloprotease type 9 (MMP-9), which is an enzyme that is known for degrading components of the extra cellular matrix (ECM) providing the structure of the dermis, under the basal membrane of the epidermis, especially collagen (Seltzer et al., 1989), and for participating in aging of the skin. UV stress increases the expression of MMP-9. Inhibition of expression of the gene responsible for the production of MMP-9, mediated by the compounds in accordance with the invention, brings about a slowing-down of degradation of the structural proteins of the dermis, preserving the integrity of the dermis over time for longer.

According to particular embodiments, the invention also satisfies the following features, implemented separately or in each of the technically operative combinations thereof.

In particular embodiments of the invention, Z represents a spacer arm bearing an amide function. Preferentially, the compound then corresponds to formula (II):

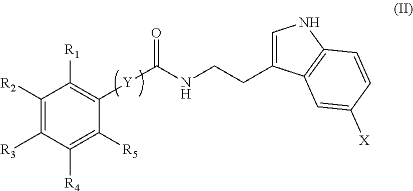

in which Y represents a covalent bond or a linear or branched, saturated or unsaturated C1-C16 hydrocarbon-based radical.

Preferentially, in this formula (II), at least one from among R₃, R₄ and R₅ represents a hydroxyl group, X represents a hydroxyl group or a methoxy group, and Y represents a covalent bond or a C2-C4 alkenyl radical.

Thus, in particular embodiments of the invention, a compound in accordance with the present invention corresponds to the general formula (III) below:

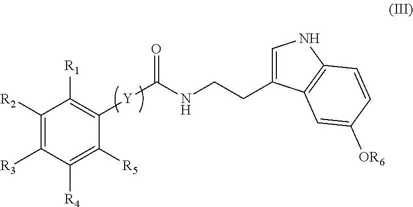

in which:

R₁, R₂, R₃, R₄ and R₅, which may be identical or different, each represent a hydrogen atom, a halogen atom, such as a fluorine, chlorine, bromine or iodine atom, a hydroxyl group or a radical —OR′ in which R′ represents a linear or branched, saturated or unsaturated C1-C16 hydrocarbon-based radical, at least one group from among R₃, R₄ and R₅ representing a hydroxyl group,

R₆ represents a hydrogen atom or a methyl group,

and Y represents a covalent bond or a C2-C4, preferably C2, alkenyl radical.

Preferentially, in formula (III) above, at least R₁ and R₂ each represent a hydrogen atom.

Compounds that are particularly advantageous in the context of a cosmetic use, in terms of efficacy of stimulation of the anti-aging cellular functions of the skin, correspond, respectively, to formula (IIa):

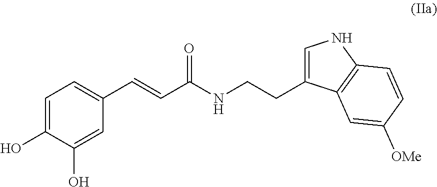 [SMILES: COc1ccc2[nH]cc(CCNC(=O)/C=C/c3ccc(O)c(O)c3)c2c1]

and to formula (IIb):

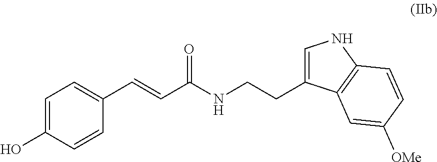 [SMILES: COc1ccc2[nH]cc(CCNC(=O)/C=C/c3ccc(O)cc3)c2c1]

The compounds in accordance with the invention may be obtained according to any method known to those skilled in the art, for example by chemical synthesis, enzymatic synthesis, by fermentation of a bacterial strain, which may or may not have been genetically modified, etc.

In particular embodiments of the invention, which are particularly advantageous in terms of efficacy of prevention and repair of the effects of aging of the skin, the compound is present in the cosmetic composition at a concentration of between 0.0000001% and 10% by weight relative to the total weight of the composition, preferably at a concentration of between 0.00001% and 1% by weight relative to the total weight of the composition.

Several of the compounds in accordance with the invention may be used simultaneously, formulated in the same cosmetic composition.

The cosmetic composition based on at least one compound in accordance with the invention is preferentially intended for topical use. The compound is present therein in a cosmetically acceptable vehicle that is standard per se. The term “cosmetically acceptable vehicle” means that the vehicle is suitable for use by being placed in contact with human and animal cells, in particular skin cells. Preferably, this vehicle has a pleasant odor, color and feel, and does not give rise to any unacceptable discomfort liable to put a user off the composition.

The composition may be in any form that is standard per se, especially, but in a nonlimiting manner, in the form of a cream, pomade, milk, oil, ointment, lotion, powder, solution, gel, suspension, soap, impregnated pad, or shampoo.

It may also comprise any additive that is standard per se in cosmetics, such as a diluent, a preserving agent, a stabilizer, emulsifier, adjuvant, carrier, etc.

The desired cosmetic effect may be reinforced by the use in the composition of any other additional active ingredient, having a beneficial effect for the skin, this effect optionally being exerted in a synergistic manner with that of the compound in accordance with the invention. Such an additional active ingredient may especially have an activity on reducing wrinkles, increasing the moisturization or firmness of the skin, reinforcing its barrier function, thickening the skin, etc. As an active ingredient that may be used in combination or association with a compound in accordance with the invention, examples that may be mentioned include anti-aging agents, antiwrinkle agents, desquamating agents, moisturizers, depigmenting agents, propigmenting agents, anti-glycation agents, NO-synthase inhibitors, agents for stimulating the synthesis of dermal or epidermal macromolecules and/or for preventing the degradation thereof, agents for stimulating the proliferation of fibroblasts and/or keratinocytes or for stimulating the differentiation of keratinocytes, muscle relaxants, dermo-decontracting agents, tensioning agents, antipollution agents and/or free-radical scavengers, anti-irritant agents, agents acting on microcirculation, agents acting on the energy metabolism of cells, anti-UV agents, and mixtures thereof, such a list not being in any way limiting.

Such agents may be chosen from carotenoids (for example beta-carotene, lycopene, astaxanthine, phytoenes), retinoids (for example retinol, vitamin A, cis or trans retinoic acid, retinol esters), flavanones, flavonols, isoflavones (for example genistein, daidzein, rutin, etc.), coumarins, lignans, vitamins (for example A, B, C, E, F, K, H), stilbenoids, sapogenins, pentacyclic triterpenic acids, β-hydroxy acids, hydroxyphenols and ether, ester or heteroside derivatives thereof, phenolic acids, tannin precursor monomers, amino sugars, amino acids (for example arginine, lysine, tyrosine, cysteine, taurine, etc.), peptides (for example carnosine, enkephalins, commercial peptides proposed for their anti-aging effect, such as Pal-KTTKS (Matrixyl® from the company Sederma), Ac-Hexapeptide 3 (Argireline from the company Lipotec), Pal-GQPR (Rigin® from the company Sederma), Dermican, Ac-tetrapeptide 9 (from BASF Beauty Solutions), Synake, tripeptide (from DMS/Pentapharm), and mixtures thereof, etc.

Another aspect of the invention relates to a cosmetic composition for stimulating the anti-aging cellular functions of the skin, comprising a compound of general formula (I):

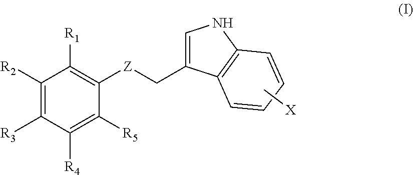

in which:

R₁, R₂, R₃, R₄ and R₅, which may be identical or different, each represent a hydrogen atom, a halogen atom such as a fluorine, chlorine, bromine or iodine atom, a hydroxyl group or a radical —OR′ in which R′ represents a linear or branched, saturated or unsaturated C1-C16 hydrocarbon-based radical, at least one group from among R₁, R₂, R₃, R₄ and R₅ not representing a hydrogen atom or a halogen atom,

X represents a halogen atom, a hydroxyl group, a nitro group, a linear or branched, saturated or unsaturated, optionally substituted C1-C14 hydrocarbon-based radical, or a radical —OR″ in which R″ represents a linear or branched, saturated or unsaturated, optionally substituted C1-C14 hydrocarbon-based radical,

Z represents a covalent bond or a spacer arm,

or a salt thereof,

in a cosmetically acceptable vehicle.

This composition may satisfy one of the features mentioned above. In particular, the compound may correspond to one of the formulae (II) or (III) above.

A process for using this cosmetic composition envisages the topical application, to the concerned parts of the body, for example to the face, of a given amount of composition, at a rate of once or twice daily, for example morning and evening, for a period of between two weeks and two months or more.

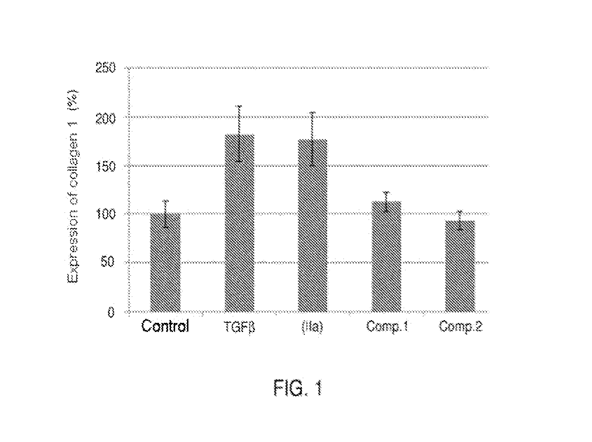

The characteristics and advantages of the invention will emerge more clearly in the light of the implementation examples below, which are given simply as illustrations of the invention that are in no way limiting, with the support of FIG. 1, which is a bar graph showing the expression of collagen 1 by normal human dermal fibroblast cells, after in vitro incubation in the absence (control) or in the presence of the compounds: TGFβ (positive control), compound in accordance with the invention (IIa), comparative compounds Comp.1 and Comp.2, the control being taken to 100%.

### EXAMPLES 1-8

### Chemical Synthesis of Compounds in Accordance with Embodiments of the Invention and of Comparative Compounds

Synthesis protocol

For all the Examples below, the synthesis protocol is as follows.

A mg of component A, and then D mg of component D are successively weighed out in a round-bottomed flask into which has been placed a magnetic bar. The flask is placed under an inert atmosphere.

In parallel, B mg of component B, and then C mg of component C are weighed out in a second round-bottomed flask into which has been placed a magnetic bar. The flask is also placed under an inert atmosphere.

F mL of anhydrous dimethylformamide (DMF) are then placed in the first flask. E mL of distilled dichloromethane (DCM), and then G μL of triethylamine (Et₃N) are added to the second flask. The two flasks are then stirred magnetically in an oil bath at 50° C. for 1 hour, and the contents of the second flask are then added rapidly by syringe into the first flask. The mixture is then refluxed on a bath at 50° C.

The reaction is monitored by thin-layer chromatography (TLC) (eluant: 7.5EA/1MeOH/1.5chloroform+1.5% trifluoroacetic acid (TFA); revelation at 254 nm and then with ninhydrin).

After 3 hours of reaction, the solution is allowed to cool to room temperature.

50 mL of ethyl acetate (EA) are then added to the crude reaction product. Three successive extractions are performed with 20 mL of aqueous 1M hydrochloric acid (HCl) solution, followed by washing with 20 mL of saturated sodium carbonate (NaHCO₃) solution and then with 20 mL of H₂O. The organic phase is recovered and concentrated under reduced pressure. 10 mL of methanol (MeOH) are added and the mixture is concentrated again under reduced pressure.

Purification is performed by column chromatography on 70-200 μm, silica, eluting with: Eluant No. 1: EA; Eluant No. 2: (7.5EA/1MeOH/1.5chloroform) +1.5% TFA; Eluant No. 3: (9EA/1MeOH) +3% TFA.

The fractions of interest are recovered and combined, and 20 mL of EA are added thereto. Four extractions are performed with 20 mL of saturated NaHCO₃ solution, followed by washing with 2×20 mL of H₂O. The organic phase is recovered and concentrated under reduced pressure. 10 mL of ethanol (EtOH) are added and the mixture is concentrated again under reduced pressure, to obtain the desired compound.

### EXAMPLE 1

### (2E)-3-(3,4-Dihydroxyphenyl)-N-[2-(5-methoxy-1H-indol-3-yl) ethyl]-2-propenamide

This compound in accordance with the invention, of formula (IIa):

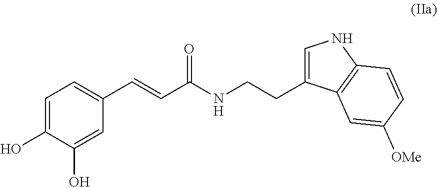

in which R₁, R₂, R₅ each represent a hydrogen atom, R₃ and R₄ each represent a hydroxyl group, X represents a methoxy group and Y represents an ethylenyl radical, is prepared as indicated in the above protocol, with the components in the following amounts:
Component A: caffeic acid/500 mg
Component B: methoxytryptamine (97%)/847 mg
Component C: 1-ethyl-3-(3-dimethylaminopropyl)carbodiimide (EDC.HCl) (98%)/846 mg
Component D: hydroxybenzotriazole (HOBt) (98%)/464 mg
Distilled DCM: Volume E=12.4 ml
Anhydrous DMF: Volume F=6.2 ml

Et₃N: Volume G=970 μl

586 mg of a brown solid are finally recovered (M=352.39 g.mol⁻¹; tr(LCMS)=5.2 min; ρ=60%; LC purity>95%).

The chromatographic analysis method (LCMS) is the following: C18 XBridge Waters 150×4.6 mm 5 μm column; eluants: water+0.1% formic acid (HCOOH)/acetonitrile+0.1% HCOOH; flow rate 1 ml/min; start of the gradient: 95-5, after 9 min: 5-95, and then isocratic gradient for 30 seconds: 5-95, and return to 95-5 over 1 min. Detection by UV detector.

Proton (¹H) NMR analysis: 3.605 (1, 3H), 6.528 (5, 1H, d, J=15.688), 7.502 (6, 1H, d, J=15.688), 7.657 (7, 1H, dd, J=8.447, J=1.944), 6.763 (8, 1H, dd, J=8.447, J=1.177), 6.754 (9, 1H, dd, J=8.451, J=1.651), 7.020 (10, 1H, ddd, J=8.451, J=5.495, J=5.376), 7.286 (11, 1H, dd, J=5.495, J=4.570), 7.253 (12, 1H, dd, J=1.944, J=1.177), 7.114 (13, 1H, ddd, J=5.376, J=4.570, J=1.651), 3.478 (14, 2H, t, J=6.356), 2.913 (15, 2H, t, J=6.356)

### EXAMPLE 2

The compound in accordance with the invention of formula (IIc):

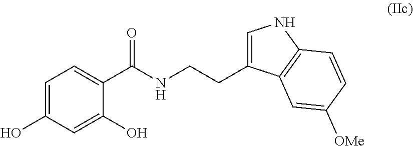

in which R₁, R₂ and R₄ each represent a hydrogen atom, R₃ and R₅ each represent a hydroxyl group, X represents a methoxy group and Y represents a covalent bond, is prepared as indicated in the above general protocol, with the components in the following amounts:
Component A: 2,4-dihydroxybenzoic acid/48 mg
Component B: methoxytryptamine (97%)/80 mg
Component C: EDC.HCl (98%)/92 mg
Component D: HOBt (98%)/58 mg
Distilled DCM: Volume E=2.5 ml
Anhydrous DMF: Volume F=1.5 ml
Et₃N: Volume G=0.1 ml

65.8 mg of a solid are recovered (M=326.4 g.mol⁻¹; tr(LCMS)=7.5 min; ρ=70%; LC purity>95%)

Proton NMR analysis: 3.605 (1, 3H), 6.942 (5, 1H, dd, J=8.099, J=2.467), 7.613 (6, 1H, dd, J=8.099, J=0.494), 6.815 (7, 1H, dd, J=8.497,

J=1.622), 6.942 (8, 1H, ddd, J=8.497, J=2.756, J=1.268), 7.371 (9, 1H, dd, J=5.280, J=2.756), 6.515 (10, 1H, dd, J=2.467, J=0.494), 7.114 (11, 1H, ddd, J=5.280, J=1.622, J=1.268), 3.507 (12, 2H, t, J=6.369), 2.915 (13, 2H, t, J=6.369)

### EXAMPLE 3

The compound in accordance with the invention of formula (IId):

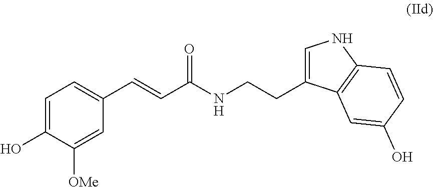

in which R₁, R₂ and R₅ each represent a hydrogen atom, R₃ represents a hydroxyl group and R₅ represents a methoxy group, X represents a hydroxyl group and Y represents an ethylenyl radical, is prepared as indicated in the above general protocol, with the components in the following amounts:
Component A: ferulic acid/33 mg
Component B: serotonin, HCl (97%)/51 mg
Component C: EDC.HCl (98%)/54 mg
Component D: HOBt (98%)/28 mg
Distilled DCM: Volume E=1.5 ml
Anhydrous DMF: Volume F=0.5 ml
Et₃N: Volume G=0.08 ml

40 mg of a brown solid are finally recovered (M=352.14 g.mol⁻¹; tr(LCMS)=6.6 min; ρ=68%; LC purity>95%)

Proton NMR: 3.784 (1, 3H), 6.528 (5, 1H, d, J=15.683), 7.514 (6, 1H, d, J=15.683), 7.588 (7, 1H, dd, J=8.449, J=1.944), 6.776 (8, 1H, dd, J=8.449, J=0.644), 6.780 (9, 1H, dd, J=8.440, J=1.935), 7.029 (10, 1H, ddd, J=8.440, J=5.498, J=0.631), 7.241 (11, 1H, dd, J=3.967, J=0.631), 7.229 (12, 1H, dd, J=1.944, J=0.644), 7.129 (13, 1H, ddd, J=5.498, J=3.967, J=1.935), 3.497 (14, 2H, t, J=6.274), 2.922 (15, 2H, t, J=6.274)

### EXAMPLE 4

The compound in accordance with the invention of formula (IIe):

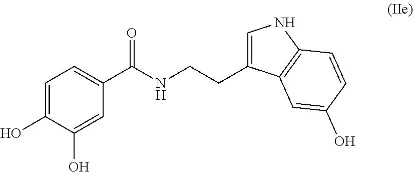

in which R₁, R₂ and R₅ each represent a hydrogen atom, R₃ and R₄ each represent a hydroxyl group, X represents a hydroxyl group and Y represents a covalent bond, is prepared as indicated in the above general protocol, with the components in the following amounts:
Component A: 2,4-dihydroxybenzoic acid/50 mg
Component B: serotonin, HCl (97%)/86 mg
Component C: EDC.HCl (98%)/94 mg
Component D: HOBt (98%)/54 mg
Distilled DCM: Volume E=2.5 ml
Anhydrous DMF: Volume F=1.5 ml
Et₃N: Volume G=0.16 ml

42 mg of a solid are finally recovered (M=312.13 g.mol⁻¹; tr(LCMS)=5.6 min; ρ=42%; LC purity>95%)

Proton NMR: 6.965 (5, 1H, dd, J=8.393, J=1.950), 7.323 (6, 1H, dd,

J=8.393, J=1.631), 6.829 (7, 1H, dd, J=8.461, J=1.284), 6.897 (8, 1H, ddd, J=8.461, J=2.432, J=1.890), 7.388 (9, 1H, dd, J=5.485, J=2.432), 7.455 (10, 1H, dd, J=1.950, J=1.631), 7.129 (11, 1H, ddd, J=5.485, J=1.890, J=1.284), 3.519 (12, 2H, t, J=6.280), 2.924 (13, 2H, t, J=6.280)

### EXAMPLE 5

The compound in accordance with the invention of formula (IIf):

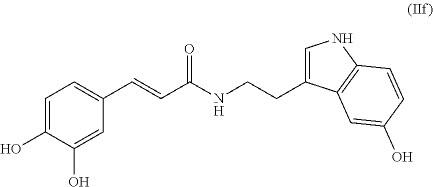

in which R₁, R₂ and R₅ each represent a hydrogen atom, R₃ and R₄ each represent a hydroxyl group, X represents a hydroxyl group and Y represents an ethylenyl radical, is prepared as indicated in the above general protocol, with the components in the following amounts:
Component A: 2,4-dihydroxycinnamic acid/60 mg
Component B: serotonin, HCl (97%)/86 mg
Component C: EDC.HCl (98%)/92 mg
Component D: HOBt (98%)/58 mg
Distilled DCM: Volume E=2.5 ml
Anhydrous DMF: Volume F=1.5 ml
Et₃N: Volume G=0.106 ml

70 mg of a solid are finally recovered (M=338.37 g.mol⁻¹; tr(LCMS)=5.9 min; ρ=65%; LC purity>95%)

Proton NMR: 6.528 (5, 1H, d, J=15.688), 7.503 (6, 1H, d, J=15.688), 7.657 (7, 1 H, dd, J=8.447, J=1.944), 6.780 (8, 1H, dd, J=8.440, J=1.935), 6.763 (9, 1 H, dd, J=8.447, J=1.177), 7.029 (10, 1H, ddd, J=8.440, J=5.498, J=0.631), 7.240 (11, 1H, dd, J=3.967, J=0.631), 7.253 (12, 1 H, dd, J=1.944, J=1.177), 7.129 (13, 1 H, ddd, J=5.498, J=3.967, J=1.935), 3.497 (14, 2H, t, J=6.274), 2.922 (15, 2H, t, J=6.274)

### EXAMPLE 6

The compound in accordance with the invention of formula (IIb):

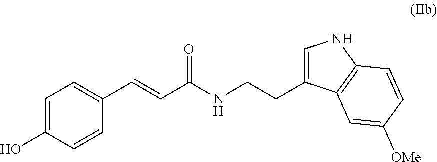

in which R₁, R₂, R₄ and R₅ each represent a hydrogen atom, R₃ represents a hydroxyl group, X represents a methoxy group and Y represents an ethylenyl radical, is prepared as indicated in the above general protocol, with the components in the following amounts:
Component A: coumaric acid/27 mg
Component B: methoxytryptamine/47 mg
Component C: EDC.HCl (98%)/47 mg
Component D: HOBt (98%)/27 mg
Distilled DCM: Volume E=1.5 ml
Anhydrous DMF: Volume F=0.5 ml
Et₃N: Volume G=0.08 ml

30 mg of a solid are finally recovered (M=336.14 g.mol⁻¹; tr(LCMS)=7.4 min; p=58%; LC purity>95%)

Proton NMR: 3.605 (1, 3H), 6.521 (4, 1H, d, J=15.574), 7.488 (5, 1H, d, J=15.574), 6.888 (6, 1H, ddd, J=8.017, J=0.445, J=0.000), 6.888 (7, 1H, ddd, J=8.017, J=0.448, J=0.000), 7.527 (8, 1H, ddd, J=8.017, J=0.448, J=0.000), 7.527 (9, 1H, ddd, J=8.017, J=0.445, J=0.000), 6.754 (10, 1H, dd, J=8.451, J=1.651), 7.020 (11, 1H, ddd, J=8.451, J=5.495, J=5.376), 7.286 (12, 1H, dd, J=5.495, J=4.570), 7.114 (13, 1H, ddd, J=5.376, J=4.570, J=1.651), 3.477 (14, 2H, t, J=6.358), 2.913 (15, 2H, t, J=6.358).

### EXAMPLE 7

### Comparative Compound Comp. 1

The comparative compound Comp.1, (2E)-3-(3-hydroxy-4-methoxyphenyl)-N-[2-(1H-indol-3-yl)ethyl]-2-propenamide, of formula (IV):

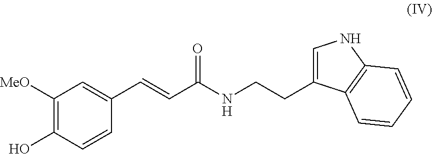 [SMILES: COc1cc(/C=C/C(=O)NCCc2c[nH]c3ccccc23)ccc1O]

in which the indole ring does not bear any substituent, is prepared as indicated in the above general protocol, with the components in the following amounts:
Component A: ferulic acid/194 mg
Component B: tryptamine (97%)/264 mg
Component C: EDC.HCl (98%)/287 mg
Component D: HOBt (98%)/162 mg
Distilled DCM: Volume E=3 ml
Anhydrous DMF: Volume F=1 ml
Et₃N: Volume G=0.35 ml

60 mg of a beige-brown solid are recovered (M=336.38 g.mol⁻¹; tr(LCMS)=9.5 min; ρ=18%; LC purity>92%)

Proton NMR analysis: 2.86 (13, 2H, t, J=7.1), 3.49 (12, 2H, t, J=7.1), 3.83 (24, 3H, s), 5.35 (7, 1H, s), 6.46 (27, 1H, d, J=15.1), 6.79 (4, 1H, dd, J=7.5, J=1.5), 6.99 (5, 1H, d, J=7.5), 7.11 (20 and 21, 2H, ddd, J=7.5, J=7.5, J=1.5), 7.16 (2, 1H, d, J=1.5), 7.32 (22, 1H, dd, J=7.5, J=1.5), 7.37 (26, 1H, d, J=15.1), 7.47 (15, 1H, s), 7.60 (19, 1H, dd, J=7.5, J=1.5), 8.03 (11, 1H, s), 10.10 (16, 1H, s)

### EXAMPLE 8

### Comparative Compound Comp. 2

The comparative compound Comp.2, 4-hydroxy-N-[2-(1H-indol-3-yl) ethyl]benzamide, of formula (V):

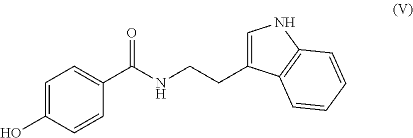 [SMILES: O=C(NCCc1c[nH]c2ccccc12)c1ccc(O)cc1]

in which the indole ring does not bear any substituents, is prepared as indicated in the above general protocol, with the components in the following amounts:
Component A: p-hydroxybenzoic acid/138 mg
Component B: tryptamine (97%)/264 mg
Component C: EDC.HCl (98%)/287 mg
Component D: HOBt (98%)/162 mg
Distilled DCM: Volume E=3 ml
Anhydrous DMF: Volume F=1 ml
Et₃N: Volume G=0.35 ml

200 mg of a beige-brown solid are recovered (M=280.32 g.mol⁻¹; tr(LCMS)=8.9 min; ρ=68%; LC purity>97%)

Proton NMR analysis: 2.90 (11, 2H, t, J=7.1), 3.55 (10, 2H, t, J=7.1), 5.35 (7, 1H, s), 6.88 (1 and 5, 1H, dd, J=7.5, J=1.5), 7.11 (18 and 19, 2H, ddd, J=7.5, J=7.5, J=1.5), 7.32 (20, 1H, dd, J=7.5, J=1.5), 7.47 (13, 1H, s), 7.60 (17, 1H, dd, J=7.5, J=1.5), 7.86 (2 and 4, 2H, dd, J=7.5, J=1.5), 8.03 (9, 1H, s), 10.10 (14, 1H, s).

### EXAMPLES 9-11

### Cosmetic Compositions Based on a Compound According to Embodiments of the Invention

### EXAMPLE 9

### Antiwrinkle Cream

An antiwrinkle day cream comprising the compound of formula (IIa), in the form of an oil (Phase A)-in-water (Phase B) emulsion, comprises the ingredients listed in Table 1 below.

TABLE 1
Constituents of the antiwrinkle cream and proportions thereof

| Constituents | Proportions | |
|---|---|---|
| Phase A | | |
| Cetearyl alcohol and Cetearyl glucoside | 5% | (w/v) |
| (Montanov ® 68) | | |
| Jojoba oil | 5% | (w/v) |
| Petrolatum oil | 5% | (w/v) |
| Isopropyl palmitate | 7% | (w/v) |
| Phase B | | |
| Glycerin | 5% | (w/v) |
| Allantoin | 0.1% | (w/v) |
| Polyacrylamide and C13-14 isoparaffin and laureth-7 | 0.3% | (w/v) |
| (Speigel ® 305) | | |
| Retinol | 0.05% | (w/v) |
| Phenoxyethanol | 0.8% | (w/v) |
| Fragrance | 0.1% | (w/v) |

| | |
|---|---|
| Compound (IIa) | 100 ppm |
| Demineralized water | qs 100% |

### EXAMPLE 10

### Body and Facial Milk

A body and facial milk comprising the compound of formula (IIb), in the form of an oil (Phase A)-in-water (Phase B) emulsion, comprises the ingredients listed in Table 2 below.

TABLE 2
Constituents of the milk and proportions thereof

| Constituents | Proportions | |
|---|---|---|
| Phase A | | |
| Oil | 5% | (w/v) |
| Stearic acid | 3% | (w/v) |
| Glyceryl stearate | 0.5% | (w/v) |
| Triethanolamine | 0.5% | (w/v) |
| Cetyl alcohol | 0.2% | (w/v) |
| Phase B | | |
| Glycerin | 5% | (w/v) |
| Caffeine | 0.5% | (w/v) |
| Carbomer | 0.2% | (w/v) |
| Methyl paraben | 0.2% | (w/v) |
| Fragrance | 0.05% | (w/v) |

| | |
|---|---|
| Compound (IIb) | 20 ppm |
| Demineralized water | qs 100% |

### EXAMPLE 11

### Moisturizing Cream

A moisturizing cream comprising the compound of formula (IIf), in the form of an oil (Phase A)-in-water (Phase B) emulsion, comprises the ingredients listed in Table 3 below.

TABLE 3
Constituents of the moisturizing cream and proportions thereof

| Constituents | Proportions | |
|---|---|---|
| Phase A | | |
| PEG-30 dipolyhydroxystearate isononanoate | 2% | (w/v) |
| (Arlacel ® P135) | | |
| Isostearyl neopentanoate | 3% | (w/v) |
| Hydrogenated polyisobutene | 3% | (w/v) |
| Octyldodecyl stearate | 9% | (w/v) |
| Ethylhexyl palmitate | 3% | (w/v) |
| Phase B | | (w/v) |
| Sorbeth-30 | 5% | (w/v) |
| Magnesium sulphate | 0.7% | (w/v) |
| Phenoxyethanol and methylparaben and ethylparaben | 0.5% | (w/v) |
| and propylparaben (Rokonsal ® MEP) | | |
| Fragrance | 0.1% | (w/v) |

| | |
|---|---|
| Compound (IIf) | 10 ppm |
| Demineralized water | qs 100% |

### EXAMPLES 12-16

### Study of the Effect of Compounds According to Embodiments of the Invention on the Expression of Skin Cell Genes

In these examples, the expression of messenger RNAs was measured via the technique of quantitative RT-PCR (reverse transcription followed by polymerase chain reaction), after in vitro incubation of the various cell types in the absence or presence of the test compounds.

This study was performed on normal human dermal fibroblasts (NHDF) or on normal human epidermal keratinocytes (NHEK), depending on the genes tested. The molecules were tested at 10⁻⁵ M in aqueous 0.1% dimethyl sulfoxide (DMSO) solution.

The cells were cultured at a concentration of 10,000 cells per well of 96-well plates, in the presence of standard culture medium as a function of the cell type.

The compounds were then added at the test concentration, for 24 hours.

The supernatant was removed and the cells were taken up in a specific buffer for extraction of the messenger RNAs (mRNA). The mRNAs were purified and reverse-transcribed in the presence of a commercial reverse transcriptase.

The complementary DNAs (cDNA) obtained were used in a real-time quantitative PCR experiment, by means of suitable primers. The degree of expression of the mRNAs was normalized with five reference genes.

### EXAMPLE 12

### Effect of Compounds on the Gene Expression of MMP-9 (Matrix Metalloprotease Type 9)

The compounds of formulae (IIa), (IIc), (IId), (IIe) and (IIf) were tested.

Caffeic acid was used as reference.

The cells used for this example were normal human keratinocytes (NHEK).

The contact with the test compounds was carried out for 24 hours.

The results obtained, in terms of decrease of the expression of MMP-9 (coded for by the gene of Genbank accession number: NG_{—}011468.1) relative to the basal level (absence of compound) taken to 100%, are shown in Table 4 below.

TABLE 4
Effect of the compounds on the expression of MMP-9

| | Decrease of expression of MMP-9 relative to |
|---|---|
| Test compounds | the basal level taken to 100% |
| Caffeic acid | No decrease |
| (IIa) | −67% |
| (IIc) | −56% |
| (IId) | −47% |
| (IIe) | −81% |
| (IIf) | −63% |

For all the tested compounds in accordance with the invention, a significant decrease in the level of expression of the protein MMP-9 is observed. Such a decrease induces a slowing-down of the degradation of the structural proteins of the dermis.

### EXAMPLE 13

### Effect of Compounds on the Expression of the Gene Coding for Sirtuin-5

The compounds of formulae (IIa), (IIb), (IIe) and (IIf) were tested.

Caffeic acid was used as reference.

The cells used for this example are normal human fibroblasts (NHDF).

The contact with the test compounds was carried out for 24 hours.

The results obtained, in terms of expression of sirtuin-5 (coded for by the gene of Genbank accession number: NC_{—}000006.11) relative to the basal level (absence of compound) taken to 100%, are shown in Table 5 below.

TABLE 5
Effect of the compound on the expression of sirtuin-5

| | Expression of sirtuin-5 relative to the |
|---|---|
| Test compounds | basal level taken to 100% |
| Caffeic acid | 100% |
| (IIa) | 153% |
| (IIb) | 125% |
| (IIe) | 146% |
| (IIf) | 140% |

For all the tested compounds in accordance with the invention, a significant increase in the level of expression of sirtuin-5 is observed. Such an increase induces an increase in cell survival, by retarding apoptosis.

### EXAMPLE 14

### Effect of Compounds on the Expression of the Gene Coding for the Protein Klotho (KL)

The compound of formula (IIb) was tested.

The cells used for this example are normal human fibroblasts (NHDF).

The contact with the test compounds was carried out for 24 hours.

The results obtained, in terms of expression of the protein Klotho (coded for by the gene of Genbank accession number: NG_{—}011485.1) relative to the basal level (absence of compound) taken to 100%, is equal to +144%. Such a significant increase in the expression of this protein brings about an improvement in the survival of these skin cells.

### EXAMPLE 15

### Effect of Compounds on the Expression of Genes Involved in Cell Differentiation and Adhesion

The compound of formula (IIa) was tested.

The cells used for this example are normal human keratinocytes (NHEK).

The contact with the test compounds was carried out for 24 hours.

The results obtained, in terms of expression of various genes relative to the basal level (absence of compound) taken to 100%, are shown in Table 6 below. In this table, after the name of each gene, the Genbank accession number is indicated in parentheses.

TABLE 6
Effect of compound (IIa) on the expression of genes
involved in cell differentiation and adhesion.

| | Gene expression |
|---|---|
| | relative to the basal |
| Genes tested | level taken to 100% |
| EPPK1 (Epiplakin 1) (NC_000008.11) | 177% |
| EVPL (Envoplakin) (NC_000017.10) | 166% |
| TGM1 (Transglutaminase 1) (NG_007150.1) | 183% |
| SPINK5 (Serine peptidase Kazal type 5) | 173% |
| (NG_009633.1) | |
| AQP3 (Aquaporin 3) (NG_007476.1) | 228% |
| CRNN (Cornulin) (NC_000001.10) | 261% |
| KRT1 (Keratin 1) (NG_008364.1) | 142% |

A significant increase in the level of expression of all the genes tested is observed. Such an increase especially promotes the formation and leaktightness of the cutaneous barrier function.

### EXAMPLE 16

### Effect of Compounds on the Expression of Genes Coding for Proteins of the Extracellular Matrix

The compound of formula (IIa) was tested.

The cells used for this example are normal human fibroblasts (NHDF).

The contact with the test compounds was carried out for 24 hours.

The results obtained, in terms of expression of various genes relative to the basal level (absence of compound) taken to 100%, are shown in Table 7 below. In this table, after the name of each gene, the Genbank accession number is indicated in parentheses.

TABLE 7
Effect of compound (IIa) on the expression of genes coding
for proteins of the extracellular matrix

| | Gene expression |
|---|---|
| | relative to the basal |
| Genes tested | level taken to 100% |
| COL1A11 (Collagen 1) (NG_007400.1) | 133% |
| COL3A1 (Collagen 3) (NG_007404.1) | 125% |
| COL5A1 (Collagen 5) (NG_008030.1) | 144% |
| ELN (Elastin) (NG_009261.1) | 136% |
| LAMA5 (Laminin alpha 5) (NC_000020.10) | 141% |
| HAS3 (Hyaluronan synthase 3) (NC_000016.9) | 144% |
| SPARC (Osteonectin) (NC_000005.9) | 118% |

A significant increase in the level of expression of all the genes tested is observed. Such an increase especially promotes the neosynthesis of proteins of the extracellular matrix.

### EXAMPLE 17

### Effect of Compounds According to the Invention and of Comparative Compounds on the Expression of Collagen 1

In this example, the expression of Collagen 1 was measured at the protein level via the immunofluorescence technique, after in vitro incubation of the cells in the absence or presence of the test compounds, at a concentration of 10 ppm in aqueous 0.1% DMSO solution.

The compound in accordance with the invention of formula (IIa) and the comparative compounds Comp.1 and Comp.2, of respective formula (IV) and (V), were tested.

TGFβ was used as positive control, at a concentration of 10 ng/ml in an aqueous solution. An untreated control was also realized.

This study was performed on normal human dermal fibroblasts (NHDF).

The cells were cultured at a concentration of 4000 cells per well in 96-well plates, in the presence of standard culture medium.

The compounds were then added at the test concentration, identical for all, for 24 hours.

The supernatant was removed and the cells were prepared for the fluorescent labeling. The cells were fixed in the presence of 3.7% formaldehyde, and then permeabilized in a buffer containing 1% Triton®, and finally the cells were rinsed and incubated in the presence of an anti-Collagen1 monoclonal antibody (Abcam) for one hour 30 minutes. After washing, the cells were incubated in the presence of the secondary antibody (Alexa GAM488, Invitrogen) for 1 hour. After washing, the cells were incubated in the presence of DAPI for 10 minutes, and then rinsed again.

The fluorescence was then quantified using an automated microscope (ArrayScan®, Cellomics®).

The results obtained, in terms of expression of Collagen 1 relative to the basal level (control) taken to 100%, are shown in FIG. 1.

It is observed that the compound in accordance with the invention (IIa) substantially stimulates the production of collagen 1 by the cells, as much as the positive control (TGFβ). On the contrary, the comparative compounds Comp.1 and Comp.2, although of similar structure to compound (IIa), do not stimulate, or only very sparingly stimulate (taking into account the standard deviation), this production of collagen 1.

The above description clearly illustrates that, by its various characteristics and the advantages thereof, the present invention achieves the objectives set. In particular, it provides compounds whose use, formulated in a cosmetic composition for topical application, acts on the functions of skin cells to increase their anti-aging action on the skin, so as to prevent, and even to repair, the symptoms of aging of the skin.

### BIBLIOGRAPHIC REFERENCES

Bradshaw A D, Reed M J, Sage EH. (2002), SPARC-null mice exhibit accelerated cutaneous wound closure, J. Histochem. Cytochem., 50(1):1-10
Chavanas S, Bodemer C, Rochat A, Hamel-Teillac D, Ali M, Irvine A D, Bonafé J L, Wilkinson J, Taïeb A, Barrandon Y, Harper J I, de Prost Y, Hovnanian A. (2000), Mutations in SPINK5, encoding a serine protease inhibitor, cause Netherton syndrome, Nat. Genet., 25(2):141-2
Contzler R, Favre B, Huber M, Hohl D. (2005), Cornulin, a new member of the “fused gene” family, is expressed during epidermal differentiation, J. Invest. Dermatol., 124(5):990-7
Eckert R L, Sturniolo M T, Broome A M, Ruse M, Rorke E A. (2005), Transglutaminase function in epidermis, J. Invest. Dermatol., 124(3):481-92
Jang S I, Kalinin A, Takahashi K, Marekov L N, Steinert P M (2005), Characterization of human epiplakin: RNAi-mediated epiplakin depletion leads to the disruption of keratin and vimentin IF networks, J. Cell Sci. 118(Pt 4):781-93
Hara-Chikuma M, Verkman A S (2005), Aquaporin-3 functions as a glycerol transporter in mammalian skin, Biol. Cell., 97(7):479-86
Karashima T, Watt F M. (2002), Interaction of periplakin and envoplakin with intermediate filaments, J. Cell Sci., 115(Pt 24):5027-37
Kuo H J, Maslen C L, Keene D R, Glanville R W. (1996), Type VI collagen anchors endothelial basement membranes by interacting with type IV collagen, J. Biol. Chem., 272(42):26522-9
Kuro-o M. (2009), Klotho and aging. Biochim. Biophys. Acta, 1790(10):1049-58
Manuskiatti W, Maibach H I. (1996), Hyaluronic acid and skin: wound healing and aging, Int. J. Dermatol., 35(8):539-44
Schlicker C, Gertz M, Papatheodorou P, Kachholz B, Becker CF, Steegborn C (2008), Substrates and regulation mechanisms for the human mitochondrial sirtuins Sirt3 and Sirt5, J. Mol. Biol., 382(3):790-801
Seltzer J L, Eisen A Z, Bauer E A, Morris N P, Glanville R W, Burgeson R E. (1989), Cleavage of type VII collagen by interstitial collagenase and type IV collagenase (gelatinase) derived from human skin, J. Biol. Chem., 264(7):3822-6
Senshu T, Kan S, Ogawa H, Manabe M, Asaga H. (1996), Preferential deimination of keratin K1 and filaggrin during the terminal differentiation of human epidermis, Biochem. Biophys. Res. Commun., 225(3):712-9
Schneider H, Mühle C, Pacho F. (2006), Biological function of laminin-5 and pathogenic impact of its deficiency, Eur. J. Cell Biol., 86(11-12):701-17
Tzaphlidou M. (2004), The role of collagen and elastin in aged skin: an image processing approach, Micron., 35(3):173-7
Uitto J. (1979), Biochemistry of the elastic fibers in normal connective tissues and its alterations in diseases, J. Invest. Dermatol., 72(1):1-10

1. The use for stimulating the anti-aging cell functions of the skin, of a compound of general formula (I): 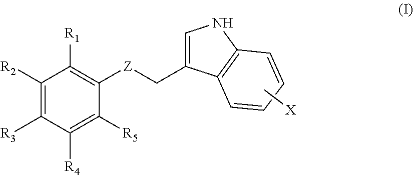
in which:
R₁, R₂, R₃, R₄, R₅, which may be identical or different, each represent a hydrogen atom, a halogen atom, a hydroxyl group or a radical —OR′ in which R′ represents a linear or branched, saturated or unsaturated C1-C16 hydrocarbon-based radical, at least one group from among R₁, R₂, R₃, R₄ and R₅ not representing a hydrogen atom or a halogen atom,
X represents a halogen atom, a hydroxyl group, a nitro group, a linear or branched, saturated or unsaturated, optionally substituted C1-C14 hydrocarbon-based radical, or a radical —OR″ in which R″ represents a linear or branched, saturated or unsaturated, optionally substituted C1-C14 hydrocarbon-based radical,
Z represents a covalent bond or a spacer arm,
or a salt thereof,
in a cosmetic composition.

2. The use as claimed in claim 1, characterized in that wherein said compound corresponds to formula (II): 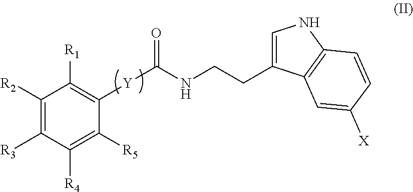
in which Y represents a covalent bond or a linear or branched, saturated or unsaturated C1-C16 hydrocarbon-based radical.

3. The use as claimed in claim 2, wherein, in formula (II), at least one from among R₃, R₄ and R₅ represents a hydroxyl group, X represents a hydroxyl group or a methoxy group, and Y represents a covalent bond or a C2-C4 alkenyl radical.

4. The use as claimed in claim 3, wherein said compound corresponds to formula (IIa):

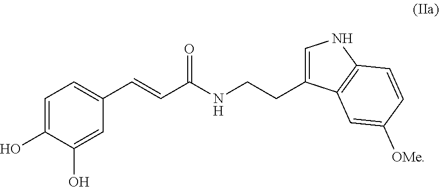 [SMILES: COc1ccc2[nH]cc(CCNC(=O)/C=C/c3ccc(O)c(O)c3)c2c1]

5. The use as claimed in claim 3, wherein said compound corresponds to formula (IIb):

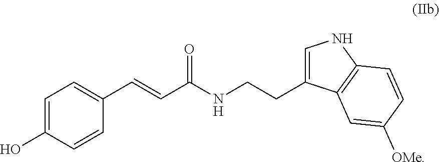 [SMILES: COc1ccc2[nH]cc(CCNC(=O)/C=C/c3ccc(O)cc3)c2c1]

6. The use as claimed in claim 1, wherein said compound is present in said cosmetic composition at a concentration of between 0.0000001% and 10% by weight relative to the total weight of the composition.

7. The use as claimed in claim 1, wherein said cosmetic composition is intended for topical use.

8. The use as claimed in claim 1, wherein said cosmetic composition is in the form of a cream, a pomade, a milk, an oil, an ointment, a lotion, a powder, a solution, a gel, a suspension, a soap, an impregnated pad, or a shampoo.

9. The use as claimed in claim 1, wherein said cosmetic composition comprises at least one additional active ingredient.

10. A cosmetic composition for stimulating the anti-aging cell functions of the skin, comprising a compound of general formula (I): 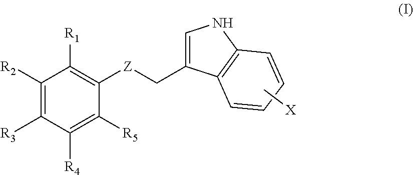
in which:
R₁, R₂, R₃, R₄ and R₅, which may be identical or different, each represent a hydrogen atom, a halogen atom, a hydroxyl group or a radical —OR′ in which R′ represents a linear or branched, saturated or unsaturated C1-C16 hydrocarbon-based radical, at least one group from among R₁, R₂, R₃, R₄ and R₅ not representing a hydrogen atom or a halogen atom,
X represents a halogen atom, a hydroxyl group, a nitro group, a linear or branched, saturated or unsaturated, optionally substituted C1-C14 hydrocarbon-based radical, or a radical —OR″ in which R″ represents a linear or branched, saturated or unsaturated, optionally substituted C1-C14 hydrocarbon-based radical,
Z represents a covalent bond or a spacer arm,
or a salt thereof,
in a cosmetically acceptable vehicle.

11. The use as claimed in claim 1, wherein said compound is present in said cosmetic composition at a concentration of between 0.00001% and 1% by weight relative to the total weight of the composition
